(19) United States
(12) Patent Application Publication
(10) Pub. No.: US 2026/0069779 A1
(43) Pub. Date: Mar. 12, 2026

(54) THRESHOLD BASED AUTOMATIC GLUCOSE CONTROL RESPONSE

(71) Applicant: INSULET CORPORATION, Acton, MA (US)

(72) Inventors: Ashutosh ZADE, San Diego, CA (US); Joon Bok LEE, Acton, MA (US); Yibin ZHENG, Hartland, WI (US)

(21) Appl. No.: 19/391,533

(22) Filed: Nov. 17, 2025

Related U.S. Application Data
(63) Continuation of application No. 17/824,494, filed on May 25, 2022, now Pat. No. 12,496,398.
(60) Provisional application No. 63/194,381, filed on May 28, 2021.

(51) Int. Cl. A61M 5/172; A61B 5/00; A61B 5/145; A61M 5/142; G16H 20/17; G16H 40/63

(52) U.S. Cl. CPC A61M 5/1723; A61B 5/14532; A61B 5/4839; A61M 5/14248; G16H 20/17; G16H 40/63; A61M 2005/14208; A61M 2205/52; A61M 2230/201

(57) ABSTRACT

Provided is a wearable medical device that includes a processor or logic circuitry. The wearable medical device may include a memory storing instructions that, when executed by the processor or logic circuitry, configure the wearable medical device to determine, by the processor or the logic circuitry, that an event affecting a blood glucose measurement value trend of a user has occurred. Based on the occurrence of the event, the processor or the logic circuitry may select a mode of operation of the analyte sensor, and generate a signal indicating the selected mode of operation. The mode of operation may correspond to a sampling frequency of a physical attribute or physiological condition of a user of the wearable medical device.

### RELATED APPLICATIONS

This application is a continuation of U.S. patent application Ser. No. 17/824,494, filed May 25, 2022, which claims the benefit of U.S. Provisional Patent Application No. 63/194,381, filed May 28, 2021, the contents of which are incorporated herein by reference in their entirety.

### BACKGROUND

Different diabetes management systems use a feedback loop control process that responds to rising, falling or steady blood glucose levels as reported by a continuous glucose monitor (CGM).

The steady and fixed interval readings from the CGM allows the control to maintain its internal state machine as well as maintain the control process's own trajectory for anticipated blood glucose trend and any anticipated bolus that may be required to make necessary corrections. However, there are pitfalls when the control process operates on a fixed time interval, such as the control process being more reactive in case of fixed CGM interval operation in responding to wider jumps in CGM values in case of meal or other events. Additionally, when the fixed interval is a large time interval, the consequence of a single “missing” CGM value causes a wider gap over which an artificial pancreas application interpolates future values. For example, when one CGM reading is missed in a 5-minute operation cycle, that one missing value translates into a 10-minute gap in CGM awareness.

It would be beneficial if blood glucose readings are processed more frequently to allow for a more rapid response by an artificial pancreas application. However, other considerations, such as power consumption and supply, affect how often readings can be provided by the CGM (as every reading and transmission of the reading consumes power) and how often each reading can be processed by a controller or a wearable drug delivery device since power is consumed every time a reading is processed.

### BRIEF SUMMARY

In one aspect, a wearable drug delivery device is provided that includes a processor, a memory, a reservoir, a pump mechanism, and a communication circuit. The memory may store an automatic glucose control application, programming code, and data related to the automatic glucose control application. The reservoir may be shaped to contain a liquid drug. The pump mechanism may be controlled by the processor and may be operable to deliver the liquid drug. The communication circuit may be controlled by the processor and operable to communicate with an external device. When the processor executes the automatic glucose control application, the processor is operable to, for a period of time, receive a blood glucose measurement value at a set time interval within the period of time. The processor may determine a rate of change of the blood glucose measurement values received from the analyte sensor over the period of time. Based on the determined rate of change, the processor may select a different set time interval.

In another aspect, a non-transitory computer-readable storage medium is provided that includes instructions executable by a processor. When the instructions are executed by the processor, the processor is operable to receive a blood glucose measurement value from an analyte sensor remote from or integrated with the wearable drug delivery device over a span of time at a set time interval. The set time interval is selected by the processor. The processor may determine a rate of change of blood glucose measurement values received from the analyte sensor over a period of time that spans multiple time intervals, and based on the determined rate of change, select a different set time interval.

In yet another aspect, an analyte sensor is provided that includes logic circuitry, a sensor, and a communication circuit. The sensor may be coupled to the logic circuitry and be operable to detect an analyte measurement value from a blood sample of a user. The sensor is further operable to make the detection of the analyte measurement value at a set detection rate. The communication circuit may be coupled to the logic circuitry and be operable to transmit a signal containing the detected characteristic of the analyte. The logic circuitry may be operable to control the sensor including setting the detection rate of the sensor based on a selection from multiple detection rates. The logic circuitry may obtain the analyte measurement value from the sensor and determine a rate of change of analyte measurement values received over a period of time. Based on the determined rate of change, the logic circuitry may determine that delivery of a liquid drug is to be suspended and may generate a suspension signal. The logic circuitry may cause the communication circuit to transmit the suspension signal.

In further aspect, a non-transitory computer-readable storage medium is provided. The computer-readable storage medium may include instructions that, when executed by a processor or logic circuitry, cause the computer to determine that an event affecting a blood glucose measurement value trend of a user has occurred. Based on the occurrence of the event, the processor or the logic circuitry may be operable to select a mode of operation of the analyte sensor, and generate a signal indicating the selected mode of operation.

In another aspect, a wearable medical device is provided that includes a processor or logic circuitry. The wearable medical device may include a memory storing instructions that, when executed by the processor or logic circuitry, configure the wearable medical device to determine, by the processor or the logic circuitry, that an event affecting a blood glucose measurement value trend of a user has occurred Based on the occurrence of the event, the processor or the logic circuitry may select a mode of operation of the analyte sensor, and generate a signal indicating the selected mode of operation.

### BRIEF DESCRIPTION OF THE DRAWINGS

To easily identify the discussion of any particular element or act, the most significant digit or digits in a reference number refer to the figure number in which that element is first introduced.

FIG. 1A shows a graphic of analyte sensor sampling at a first time interval compared to analyte sensor samples taken at a second time interval or substantially continuously.

FIG. 1B illustrates another graphic of analyte sensor sampling at a first time interval compared to analyte sensor samples taken at a second time interval or substantially continuously.

FIG. 1C illustrates timing and sampling examples related to the determination of a microbolus and sub-microbolus.

FIG. 2 illustrates a process example in accordance with one embodiment.

FIG. 3 illustrates yet another graphic of analyte sensor sampling at a first time interval compared to analyte sensor samples taken at a second time interval or substantially continuously.

FIG. 4 illustrates another process example in accordance with another embodiment.

FIG. 5 illustrates an example of a process according to an aspect of the disclosed subject matter.

FIG. 6 illustrates a functional block diagram of a system example suitable for implementing the example processes and techniques described herein.

### DETAILED DESCRIPTION

The disclosed examples provide techniques that may be used with any additional algorithms or computer applications that manage blood glucose levels and insulin therapy. These algorithms and computer applications may be collectively referred to as “medication delivery algorithms” or “medication delivery applications” and may be operable to deliver different categories of drugs (or medications), such as chemotherapy drugs, pain relief drugs, diabetes treatment drugs (e.g., insulin, glucagon, pramlintide, glucagon-like peptides, or combinations thereof), blood pressure medication, or the like.

A type of medication delivery algorithm (MDA) may include an “artificial pancreas” algorithm-based system, or more generally, an artificial pancreas (AP) application. For ease of discussion, the computer programs and computer applications that implement the medication delivery algorithms or applications may be referred to herein as an “AP application.” An AP application may be configured to provide automatic delivery of insulin or other diabetes treatment drugs based on signals received from an analyte sensor, such as a continuous blood glucose monitor (CGM), or the like. In an example, the artificial pancreas (AP) application may operate in cooperation with an automatic glucose control (AGC) application or algorithm. The AGC application when executed by a processor may enable monitoring of a user's blood glucose measurement values, determine an appropriate level of insulin for the user based on the monitored glucose values (e.g., blood glucose concentrations or blood glucose measurement values) and other information. Either alone or in cooperation with the AP application, the AGC application may be operable to maintain a user's blood glucose levels in range of a target blood glucose setting. The “target blood glucose setting” may be a setting that the AGC uses as an optimal blood glucose measurement value and performs different functions to maintain the user's blood glucose as close as possible to the setting. For example, a target blood glucose measurement value may be acceptable if it falls within the range of 110 mg/dL to 150 mg/dL, which is a range satisfying a clinical standard of care for treatment of diabetes as such the user's target blood glucose setting may be 120 mg/dL. In addition, an AGC application as described herein may be operable to determine when a user's blood glucose is getting into the hypoglycemic range (e.g., <70 mg/dL) or the hyperglycemic range (e.g., >180 mg/dL).

A system component for maintaining the user's blood glucose measurement values near the user's target blood glucose setting is a CGM. CGMs of today deliver at fixed intervals; however, eventually CGMs may be capable of performing more on-demand and more granular blood glucose measurements.

The advantages of the opportunity presented by receiving a blood glucose reading more frequently than every 5 minutes or at variable rates, such as 1 minute for a period of time (e.g., an hour), then 3 minutes for a next period, and then 5 minutes for another period or for the rest of the day or night, enables an AGC application to obtain more accurate and more frequent blood glucose readings, respond to readings more quickly always or during certain periods, thereby consuming less power. Receiving or sampling more frequently may also allow an AGC application to provide additional considerations such as triggering the more frequent reception or sampling upon certain thresholds. Moreover, in situations when there are missing readings from the CGM, a blood glucose measurement value from a most recent prior reading can be used by the AGC application to make delivery and/or sampling frequency determinations. As such, the capability to select a sampling frequency may be also be very effective to mitigate the effect of the missed reading.

By incorporating more precise and frequent readings, the AGC application can be more effective in predicting hypo- or hyper-glycemia, alert the user sooner, suspend or elevate drug deliveries more quickly, and not only improve time in range but add more safety for the user. For example, a wearable drug delivery device may be operable to control how often a CGM samples. If an AGC application executing on the wearable drug delivery device or a controller determines there is a reason for concern (e.g., a blood glucose measurement was not received, or the user is approaching or has entered a hypo- or hyper-glycemic range), the AGC application may issue a command signal to the CGM to begin sampling more frequently.

A difference two minutes, for example, when a user is experiencing an extreme negative rate of change of blood glucose measurement values, particularly when the user's blood glucose measurement values are close to the low blood glucose thresholds, can be the difference between a user being able to treat themselves and a user needing assistance or, perhaps, hospitalization.

As analyte sensor technology, and in particular, technology in wearable CGMs, advances, a processor in a wearable drug delivery device (shown and described with reference to a later example) may be operable to transition to obtaining more frequent blood glucose readings from the CGM. For example, the processor may be operable to select how often the processor should receive a blood glucose reading from the CGM. The processor may be further operable to apply the more frequently obtained blood glucose readings to determining how much medication (also referred to as “diabetes treatment drug,” “liquid drug,” “insulin,” and “therapeutic drug” herein) should be delivered to the user and when. Alternatively, the processor may be able to more quickly and more precisely determine when a user is in danger of a hypo- or hyper-glycemic event and be able to halt or increase insulin delivery sooner.

Furthermore, the time needed by a processor in a wearable drug delivery device or controller to derive a CGM value with high confidence may be further reduced because further calculations are not needed since the extra samples reduces the number of missing measurements, errors in measurements and the like However, different conditions may affect the readings of the CGM, so at times the CGM may not be as accurate as other times, or as time goes on the accuracy of the CGM may diminish at an unknown rate and in a nonlinear manner, in which case, the processor may need additional time to derive a CGM value with the high confidence level.

The possibility of on-demand readings from a CGM may be needed in certain scenarios. The on-demand reading may be more helpful in events such as a consumption (or imminent consumption) of a meal, participation in exercise, or imminent hypoglycemia. The process 200 provides an example of how a processor of the wearable drug delivery device may determine the need for more frequent readings from the CGM. Of course, another processor, such as a processor in the CGM or a controller device in an automatic medication delivery system, may be operable to receive and process blood glucose measurement values from a CGM.

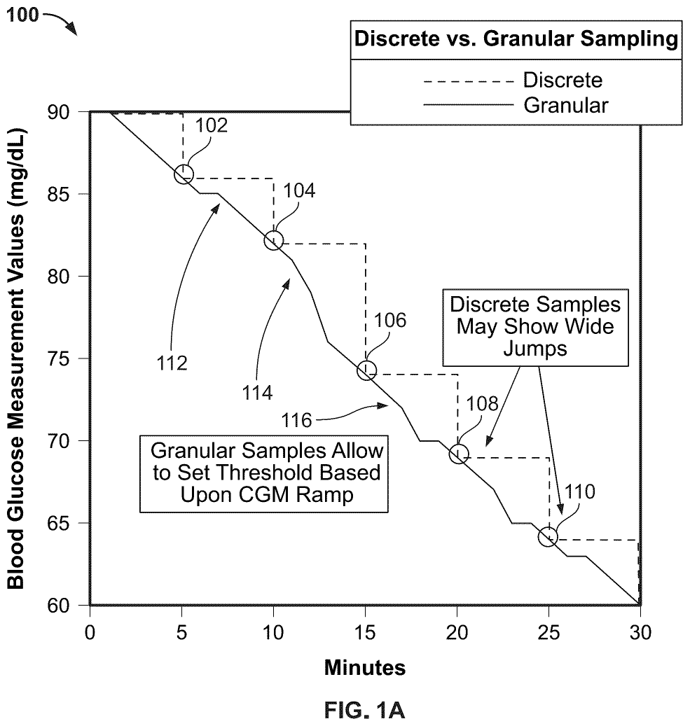

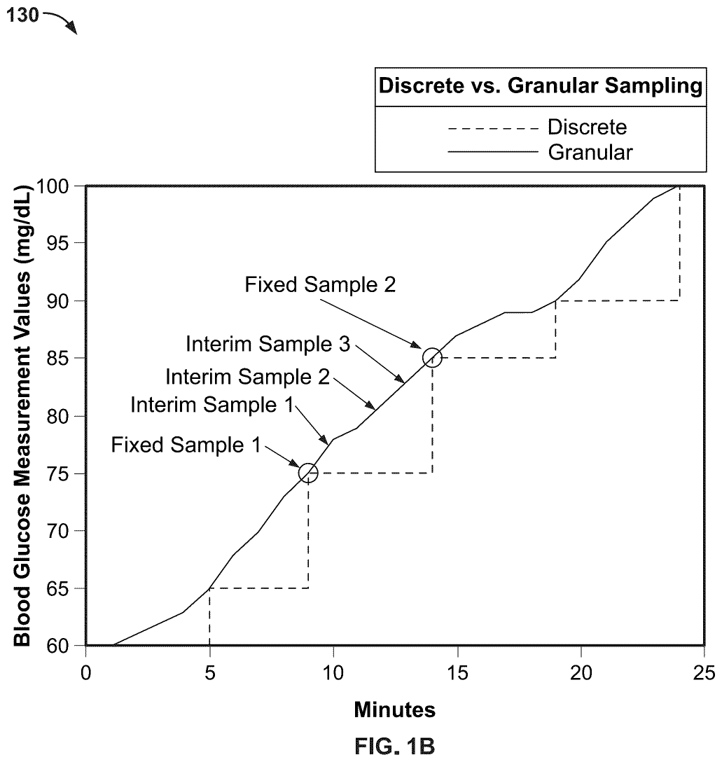

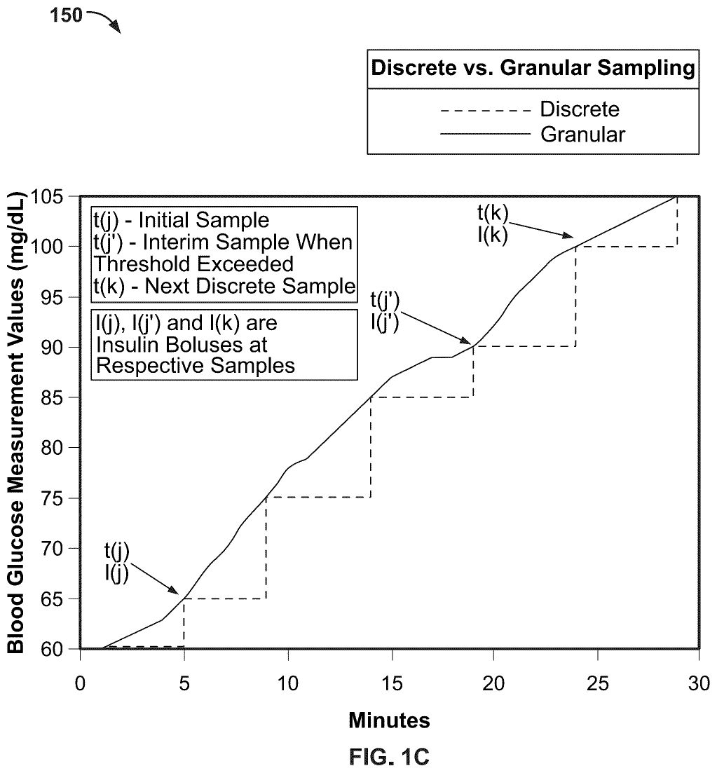

FIG. 1A shows a graphic of sampling at a first time interval in comparison to samples taken at second time interval or substantially continuously. The chart 100 has blood glucose measurement values on the vertical axis ranging from 60 mg/dL to 90 mg/dl and time on the horizontal axis ranging from 0 to 30 minutes.

In the example shown in FIG. 1A, the processor may receive a blood glucose measurement value at the set time interval of every 5 minutes. For example, at samples 102, 104, 106, 108, and 110, blood glucose measurement value of approximately 86, 83, 74, 68, and 63, respectively, were obtained at 5-minute intervals. The samples 102-110 are shown on a stair-step line (top dashed line). This time interval of 5 minutes has been shown to consume power efficiently and allows for an appropriate reserve power, such as 10-20%, for contingency operations (e.g., additional processing time, communications, alerts or notifications, or the like). Chart 100 illustrates how sampling at the first interval, e.g., 5 minutes, slowly reveals a negative rate of change in blood glucose measurement values that may lead to a user's blood glucose falling below a hypoglycemic threshold, such as 60 mg/dL or 70 mg/dL.

Alternatively, or in addition, the analyte sensor or CGM may be operable to obtain blood glucose measurement values at smaller or more granular time intervals (e.g., every 1 minute compared to every 5 minutes), such as those shown by the lines 112, 114 and 116, which represent a trend of a number of blood glucose measurement values received or sampled, for example, every one minute.

FIG. 1B illustrates another graphic of analyte sensor sampling at a first time interval compared to analyte sensor samples taken at a second time interval or substantially continuously. In situation in which the blood glucose is exhibiting a rising trend (i.e., a positive rate of change) as illustrated in the chart 130 of FIG. 1B, the AGC application may benefit from obtaining CGM blood glucose measurement values sooner than at a fixed reading rate (such as that which may be provided by fixed sample 1 and fixed sample 2 because the AGC application may be able to deliver sub-microboluses which can avoid more reactive microboluses. A microbolus may be a dosage of insulin between 0.1-0.425 Units of insulin, while a sub-microbolus may be approximately 0.05-0.15 Units of insulin. The determination of a microbolus and sub-microbolus is described in more detail with reference to a later example. For example, the disclosed process provides an additional benefit of mitigating the effects of an estimated reading when there are missing blood glucose data points (for example, if fixed sample 2 was not received by a controller) or increasing blood glucose data points. For example, in the case of a rising blood glucose trend, the AGC can deliver a sub-microbolus sooner, such as a 0.3 mg/dL of medication. In example of FIG. 1B, if X is a future reactive micro-bolus to be delivered after interim sample 3, the AGC may apply threshold-based decisions using the values of interim sample 1 and interim sample 2 between fixed sample 1 and fixed sample 2 to determine a fast rising trend in the blood glucose. As a result of the determination, the AGC may determine that it is appropriate to deliver a sub-microbolus, which may be a medication dosage approximately equal to X/2, where X is considered as the reactive micro-bolus. By delivering more sub-boluses sooner, the future rising trend may be improved (i.e. reduced to become less steep or even plateau) sooner.

FIG. 1C illustrates timing and sampling examples related to the determination of a microbolus and sub-microbolus. As mentioned above, a microbolus may be a dosage of insulin between 0.1-0.425 U of insulin, while a sub-microbolus may be approximately 0.05-0.15 Units of insulin. The determination of a microbolus and sub-microbolus is described in more detail with reference to a later example.

The amount of insulin dose for a microbolus or sub-microbolus may be derived by considering IOB and IOB constraint is described in the following paragraphs.

The IOB required for a user may be determined according to Equation 1.

Equation (1) below is a modified version of a standard correction bolus calculation. Specifically, the amount of IOB required at a given time-step k (i.e., IOB_{required}(k)) is computed from the difference between the CGM value at time-step k (CGM(k)) and the glycemic setpoint (Gₛₚ) divided by the insulin correction factor (CF). The IOB required (IOB_{required}(k)) is computed as follows:

$${IOB}_{required} ( k ) = \frac{CGM ( k ) - {G}_{sp}}{CF} if CGM ( k ) > {G}_{sp} , \qquad ( Eq 1 )$$

$$where {IOB}_{required} ( k ) = 0 if CGM ( k ) \leq {G}_{sp} , and {G}_{sp}$$

is the blood glucose set point.

An amount of IOB may be constrained using an IOB constraint. Subsequently, the IOB constraint I_{IOB}(k) at time-step k is the IOB required (IOB_{required}(k)) minus the IOB in units of insulin per time-step k computed as follows:

$${I}_{IOB} ( k ) = {IOB}_{required} ( k ) - IOB ( k ) + \frac{1}{12} {I}_{basal}^{hourly} if {IOB}_{required} > IOB , \qquad ( Eq . 2 )$$

$$where {I}_{IOB} ( k ) = \frac{1}{12} {I}_{basal}^{hourly} if {IOB}_{required} \leq IOB .$$

In some examples, there may not be constraints on the AGC algorithm or application that limit the delivery of insulin below basal insulin at any given time-step k, except for a hard coded safety constraint that suspends insulin delivery at glucose below 60 mg/dL regardless of any other factors. Other than the hard-coded suspension below 60 mg/dL, any delivery less than the basal is the result of the minimization of a cost function utilized by the AGC algorithm or application.

Referring to chart 150 of FIG. 1C, at any such point, say at k, I(k)≤I_{IOB}(k). Likewise, the suggested dose at I(j) may be based upon CGM(j) and IOB(j).

An additional data point at sample (j′) in which the threshold (i.e., the granular reading continues to increase) is breached as a 3^{rd} consecutive sample of granular reading, the AGC algorithm or application may derive I(j′) using similar equations.

At any such interim point t(j′), the threshold may be breached. For example, I(j′)<I(k), where CGM(k)>CGM(j′).

The suggested insulin dosages to be delivered as micro-boluses at I(j) and I(k) may be based upon discrete sampling. For example, since, I(j′)<I(k), at 3^{rd} consecutive CGM sample in which the blood glucose trend is rising, the AGC algorithm may cause delivery of a sub-microbolus as (I(k)/5)*3. For example, a microbolus calculated as (I(k)/5)*3 may have an approximate value between 0.1 and 0.425 Units of insulin. While I(k) is considered a more reactive micro-bolus, I(j′) is sooner and avoids reactive bolusing at t(k) due to granular availability of CGM(j′) earlier.

In the above equations, the symbol I(k) has the units U and represents insulin delivery at a given time-step k (e.g., units U/5 min). The symbol I′(k) has the units U and represents an insulin deviation, which is the difference between an amount of insulin requested in an insulin dose command from the AGC algorithm and pre-programmed basal rate. The symbol

$${I}_{h i}^{′} ( k )$$

has units U and represents a positive deviation of insulin delivery above pre-programmed basal rate at time-step k, where U is defined as units of insulin per 5 minutes). The symbol

$${I}_{l o}^{′} ( k )$$

is in units U and represents a negative deviation of insulin delivery below pre-programmed basal rate at time-step k. In this example, insulin is constrained ≥0. The symbol CGM(k) is in units of mg/dL, which is a CGM measurement at time-step k. The symbol Gₚ(k) is in units of mg/dL and represents a predicted blood glucose at time-step k. The symbol G′ (k) is in units of mg/dL and represents a deviation of glucose G from glycemic setpoint Gₛₚ, e.g., G′ (k)=Gₚ(k) −Gₛₚ(k) for predicted glucose Gₚ. The symbol I_{IOB}(k) is in units U and represents an IOB constraint on the insulin delivery at time-step k. The symbol I_{AGC}(k·l) is in units U and represents a dose of insulin recommended by that algorithm at time (k·l), where/is value less than or greater than a time-step k. The symbol I_{basal} hourly is in units U and represents an insulin basal rate per hour.

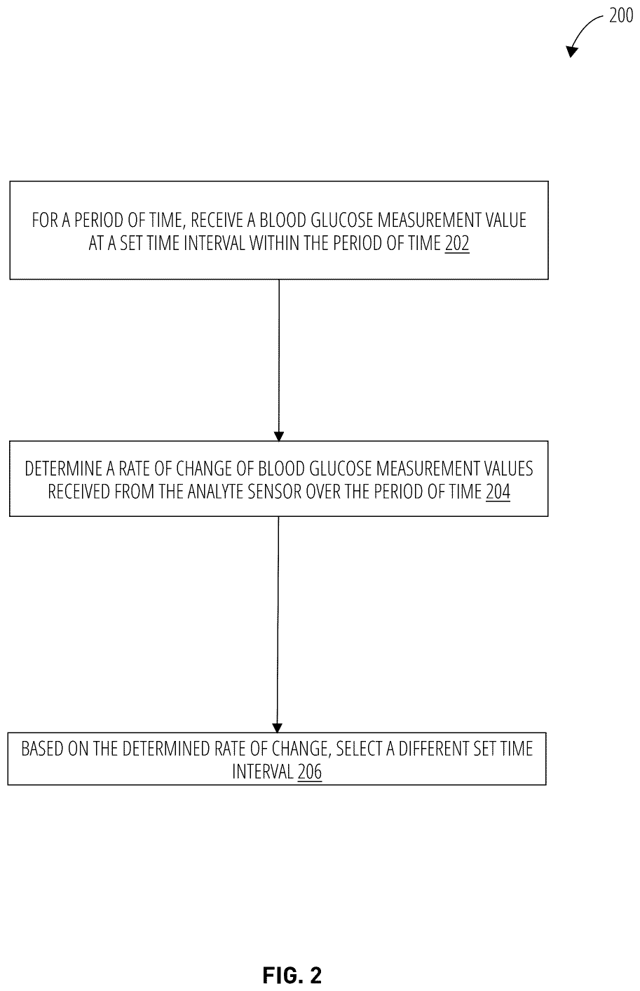

The AGC algorithm can also be invoked based upon crossing thresholds. A simple threshold establishment can be done as described with reference to the example process illustrated in FIG. 2.

FIG. 2 illustrates a process in accordance with one embodiment. The process 200 may be implemented by a processor of a wearable drug delivery device, or by a logic circuitry of CGM(shown in a later example).

In block 202, the processor of the wearable drug delivery device executing process 200 may be operable to receive data, such as a blood glucose measurement value, from an analyte sensor remote from or integrated with the wearable drug delivery device at set time interval and may receive a number of blood glucose measurement values over a period of time. The set time interval may, for example, be selected by the processor and the period of time may span multiple set time intervals, such as 15 minutes, 30 minutes, 60 minutes, 90 minutes, 120 minutes, or the like.

In block 204, the processor determines a rate of change of the blood glucose measurement values received from the analyte sensor over a period of time that may span multiple time intervals. For example, the CGM may output a blood glucose measurement value at time 102. This processor may identify the blood glucose measurement value associated with time 102 as CGM(t=0)), but as time proceeds and the next blood glucose measurement value is received at time 104, the processor may relabel the blood glucose measurement value at time 102 as CGM(t=(−)1) and store the relabeled blood glucose measurement value in a memory. This relabeling and setting of the present blood glucose measurement value as CGM(t=0) and decrementing the labels of the previously-received blood glucose measurement values may proceed as the number of samples increases and present blood glucose measurement values are received by the wearable drug delivery device and stored in a memory.

As the blood glucose measurement values are received and stored, the processor, when determining the rate of change of blood glucose measurement values over the period of time that spans multiple time intervals, may be operable to utilize a sample threshold equation to determine whether a rate of change between the blood glucose measurement values at the different times exceeds a sample threshold value. Alternatively, the processor may be operable to utilize several select samples (e.g., blood glucose measurement values from the past five times (e.g., time=(−)4 to time=0) or the like in the sample threshold equation to determine whether a rate of change between blood glucose measurement values at different times exceeds a sample threshold value.

In an example, the processor of the wearable drug delivery device may be operable compare the blood glucose measurement values from different times (e.g., CGM (t=1) to CGM (t=0)) to determine a rate of change.

The sample threshold can be defined as shown in Equation 3 below:

$$sample threshold ( t ) = c + ( CGM ( t = X - 1 ) ) - CGM ( t = ( X ) ) / seconds$$

where X is a time when a current sample (e.g., time 104) has been taken and X−1 (e.g., time 102) is the time when a previous sample was taken, and error compensation “c” in the threshold (t) calculation of Equation 3 may be used to accommodate for errors in the CGM reading. For example, the error compensation “c” value may be an integer value, a constant value (e.g., 3.5), a variable, a percentage, such as 0.5%, 1% or the like of the total CGM difference (i.e., CGM(t=(X−1))−CGM(t=x)), or the like.

If the calculated value of the sample threshold at a particular time, such as sample threshold (t), where t=X, exceeds a certain value, the processor may interpret the calculated sample threshold (t) value as indicating a rapid decrease (e.g., 9 or more mg/dL per minute or the like) in the user's blood glucose measurement levels.

In block 206 of process 200, based on the determined rate of change, the processor may be operable to select a different set time interval for receiving blood glucose measurement values.

In one example, the processor may be operable to choose the different set time interval from a table when selecting the different set time interval. For example, the table may be based on the determined rate of change and each different set time interval may be a set time value that, for example, is half, one third, one quarter or one fifth of the set time interval. Of course, other set time intervals may be included in the table of different set time intervals.

In another example, the processor when selecting the different set time interval may be operable to calculate the different set time interval based on the determined rate of change and the set time interval by dividing the set time interval by a constant. In addition, the processor may be further operable to, after selection of the different set time interval, set the different set time interval to the set time interval for future receipt of blood glucose measurement values at the set time interval.

In a further example, an action that the processor may be operable to take is to automatically increase the number of CGM readings. For example, the processor may determine that based on the blood glucose measurement values at samples 102 and 104, or a rate of change thereof, exceeding the sample threshold, the set time interval should be 1, 1.5, 2.0, 2.5, 3.0 minutes or the like. As a result, the processor may start receiving blood glucose measurement values from the CGM at a number of times such as those represented by lines 114 and 116 and, which may include a time that corresponds to sample 106. In the example of FIG. 1A, the negative rate of change of the blood glucose measurement values indicates that the user may be heading toward a hypoglycemic event. By switching to a shorter or more frequent set interval, the time that the processor of the wearable drug delivery device needs to determine when to generate an alarm may be shortened and, as a result, the user may be alerted earlier and not experience the hypoglycemic event or experience reduced symptoms of the hypoglycemic event because of intervention by the processor (executing an automatic glucose control application).

In addition, or alternatively, the processor may select a different set time interval based on the CGM output settings and the wearable drug delivery device receipt settings as well as the determined rate of change. For example, the processor may consider the CGM output settings are already at their highest rate of output of blood glucose measurement values, while the wearable drug delivery device settings are at a more relaxed rate for receiving the blood glucose measurement values from the CGM. As such, in response to a negative rate of change, the processor may determine that the wearable drug delivery device settings (e.g., set time interval) are to be adjusted to increase the frequency at which the wearable drug delivery device is set to receive the more frequent outputs from the CGM. For example, the frequency at which the wearable drug delivery device is set to receive outputs from the CGM may be increased to match or closely match the output frequency of the CGM.

The processor may, after selection of the different set time interval, set the different set time interval as the set time interval for future receipt of blood glucose measurement values at the set time interval.

In another operational example, the CGM may be set to output values at an increased frequency, but the processor of the wearable drug delivery device may be operable to, via selectable settings of the wearable drug delivery device, receive the outputted blood glucose measurement values at the set interval. For example, the wearable drug delivery device may have a user interface used to set up the CGM, or a controller may have a user interface that enables, via a communication session with the CGM an initial set up of the CGM settings and the wearable device or controller settings. During the setup of the CGM, the respective user interface may enable the user (or guardian or health care provider) to select a set time interval when the wearable drug delivery device (or controller) receives the blood glucose measurement values from the CGM. Alternatively, the setting of the output of the blood glucose measurement values from the CGM may be according to a default setting of a set time interval, such as every 5 minutes. Either the user-selected set time interval or the default set time interval may correspond to the set time interval for the AGC application to receive the blood glucose measurement values from the CGM.

In more detail, the CGM, via a communication session established by a communication circuit of the wearable drug delivery device with a communication circuit of the CGM(both shown in a later example), may be operable to receive commands that cause the CGM to increase (or decrease) the frequency of sampling of blood glucose and outputting the blood glucose measurement values obtained from sampling the blood of the user. In addition, after selection of the different set time interval, the processor may replace the set time interval with the selected different set time interval for future receipt of blood glucose measurement values.

The process 200 enables the processor when executing an AGC application to suspend insulin delivery more quickly in the case of a blood glucose downward trend (i.e., a negative rate of change).

In more detail, as shown in FIG. 1A, the processor of the wearable drug delivery device may receive blood glucose measurement values at samples 102, 104, and 106 (that correspond to times at 5 minutes, 10 minutes and 15 minutes, respectively), but may not receive the blood glucose measurement value at sample 108 (which corresponds to the time 20 minutes) because of a communication error, such as a loss of connectivity or inability to establish a communication session between processor and CGM.

In another beneficial embodiment, the more rapid CGM measurements may be selected in cases where rapid rates of change may involve high risk physiological conditions. For instance, if the rate of change in the user's glucose may lead the user to go below a hypoglycemic threshold setting (e.g., 70 or 60 mg/dL) over the next 10 minutes, for example, then the processor may automatically cause receipt of blood glucose measurement values on demand from the CGM more often than an initial set time interval of 5 minutes. For example, if the user's glucose is 300 mg/dL, and the next measurement 5 minutes later is 250 mg/dL, then the rate of change (ROC) is a negative 50 mg/dL per 5 minutes (i.e., (−)50 mg/dL/5 min). The user's blood glucose may drop 100 mg/dL per 5 minutes for the next 10 minutes, potentially causing the user to experience hypoglycemia.

Although the processor may be operable to generate an alarm or an alert at the 10 minute mark, this may not be sufficiently rapid for the user to avoid symptoms of hypoglycemia. In addition, or alternatively, if the AGC application may be operable to recognize this rapid negative rate of change, the AGC application, according to the examples described herein, is operable to trigger a measurement every minute, and be able to alert for low glucose at the 8 minute mark instead, alerting the user 2 minutes earlier than the previous efforts. The processor of the wearable drug delivery device and/or the logic circuitry of the CGM may be operable to follow a rule of when the threshold for rate of change is surpassed, the frequency of sampling and delivery of the blood glucose measurement values is increased.

The processor may be able to use the blood glucose measurement values from samples 102-106 to estimate an expected blood glucose measurement value of the reading that was supposed to be received at sample 108. However, there will be a degree of uncertainty with the expected blood glucose measurement value until the CGM reading for sample 110 is received to update the estimate of the expected blood glucose measurement value for sample 108. The time gap caused by 1 missing CGM reading when the set time interval is 5 minutes is 10 minutes. For some users, a 10 minute time gap when their blood glucose measurement values are trending downward (i.e., have a negative rate of change) may be harmful to them as they may begin experiencing symptoms of a hypoglycemic event. Hence, it is an advantage to have a set time interval that is shorter than 5 minutes, but also an advantage to be able to quickly switch to a shorter set time interval. For example, the processor may be able to switch to a more frequent set time interval (sampling every 1 minute, such as times 112, 114 or 116) upon a determination that a blood glucose measurement value has been missed.

In the case of where the determined rate of change is determined to be a positive rate of change, or in other words, the blood glucose measurement values are trending upward, such as from 115 mg/dL to 130 mg/dL, the processor may take no action with regard to the set time interval. Alternatively, or in addition, the processor may be operable, in response to a determination that the determined rate of change is beginning to trend positive, to select a different set time interval that is less frequent. For example, if the set time interval is 1 minute, the processor, in response to a detected positive rate of change, may select a different set time interval that is within the capability of the CGM, such as 2 minutes, 3.5 minutes, 5 minutes, or other times. The selection of the time as the different set time interval may be based on the duration (how long the rate of change has been positive) or magnitude of the upward trend in the blood glucose measurement values.

In addition, the response of the processor to the sample threshold not being exceeded or the rate of change being low, may be to select a different set time interval that increases the time between receipt of blood glucose measurement values. In the case where the determined rate of change remains low (both in the negative range and positive range), such as approximately zero or within range of a target blood glucose level (e.g., within ±5% of a target blood glucose setting of 115 mg/dL) over a predetermined number of measurements or a time period, the processor may determine to reduce the frequency of receipt of the blood glucose measurement values from the CGM. For example, if the rate of change is low (either positive or negative), the processor may determine that the set time interval may be set back to 5 minutes. Doing so may extend the life of the CGM sensor and/or batteries within the CGM. Alternatively, the selection of different set time intervals may be in stages, for example, if the ROC surpasses Y (e.g., where Y is 10 mg/dL per minute), then switch to 1 minute; if the ROC decreases below Y, then go back to 5 minute sampling.

The wearable drug delivery device may also include a communication circuit that may be operable to establish, in response to a control signal from the processor, a communication session with an analyte sensor, such as the CGM, that may be remote from the wearable drug delivery device. The communication session is established to enable receipt of the blood glucose measurement value at the set time interval. During the communication session, the processor may receive the blood glucose measurement value as well as transmit control signals to the CGM regarding changes to the set time interval and the like. Other technical features may be readily apparent to one skilled in the art from the following figures, descriptions, and claims.

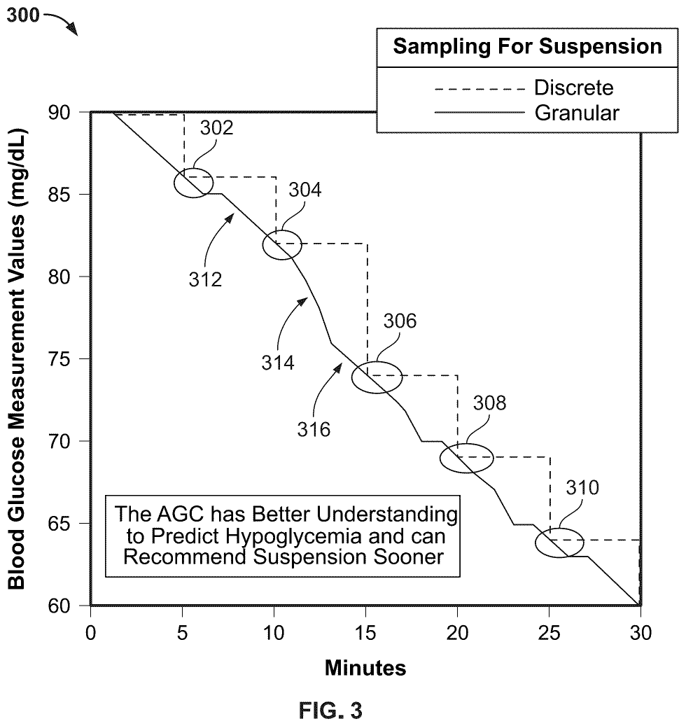

FIG. 3 illustrates another graphic of analyte sensor sampling at a first time interval compared to analyte sensor samples taken at a second time interval, which may be substantially continuously.

A variable sampling setting for both a wearable drug delivery device and a CGM may be beneficial to enable the processor executing an AGC application to make a more rapid determination to suspend delivery of insulin. The chart 300 has a vertical axis of blood glucose measurement values ranging from 60 mg/dL to 90 mg/dL and a horizontal axis of number of minutes 0 to 30.

In the example shown in FIG. 3, the processor may receive a blood glucose measurement value at the set time interval of every 5 minutes. For example, sample 302 corresponds to time 5 minutes with a blood glucose measurement value of approximately 86 mg/dL, sample 304 corresponds to time 10 minutes with a blood glucose measurement value of approximately 83 mg/dL, sample 306 corresponds to time 15 minutes with a blood glucose measurement value of approximately 74 mg/dL, sample 308 corresponds to time 20 minutes with a blood glucose measurement value of approximately 68 mg/dL, and sample 310 corresponds to time 25 minutes with a blood glucose measurement value of approximately 63 mg/dL. The samples 302-310 are shown on a stairstep (dashed) line. This time interval of 5 minutes has been shown to consume power efficiently and allows for an appropriate reserve power, such as 10-20% for contingency operations (e.g., additional processing time, communications, alerts or notifications, or the like). Chart 300 illustrates how sampling at the first interval, e.g., 5 minutes, reveals blood glucose measurement values of samples 302, 304, 306, 308 and 310 that illustrate a negative rate of change that may lead to a user's blood glucose falling below a hypoglycemic threshold, such as 60 mg/dL or 70 mg/dL.

Alternatively, or in addition, the analyte sensor or CGM may be operable to obtain blood glucose measurement values at smaller time intervals and provide them to a wearable drug delivery device or controller. The smaller time intervals and samples may be indicated by lines 312, 314 and 316, which represent a number of samples closer in time than the discrete samples 302-310. Of course, the smaller time intervals may be approximately any period of time (e.g., 30 seconds, 1 minute, 2.5 minutes, 3 minutes or the like) along the continuous line (that includes lines 312, 314, and 316).

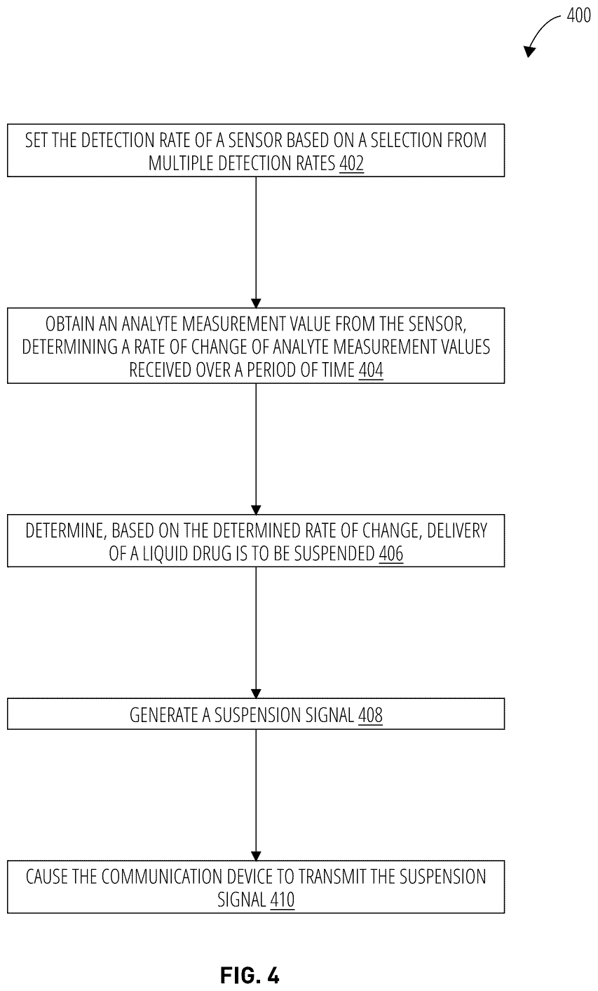

Moreover, the processor implementing the AGC application may gain better insight into the user's changing blood glucose measurement values, such as decreasing values that indicate a potential hypoglycemia event or increasing values that indicate a potential hyperglycemia event. In the case of the user's blood glucose measurement values trending downward, the processor may be able to more quickly determine that further delivery of insulin should be suspended and take the appropriate action, such as generating control signals to suspend insulin delivery, issuing an alarm, issuing a recommendation to the user to consume carbohydrates, and the like. It may be helpful to describe a process example with reference to FIG. 3 and FIG. 4 below.

FIG. 4 illustrates a flowchart of an example process according to an embodiment of the disclosed subject matter. The process 400 of FIG. 4 may be implemented by a processor or logic circuitry of an analyte sensor. An example of an analyte sensor may be a continuous glucose monitor that is operable to detect blood glucose and output a blood glucose measurement value. The analyte sensor may include logic circuitry, a sensor and a communication circuit. In the example, the logic circuitry may be disposed in an analyte sensor, such as a CGM, as well as a communication device. The logic circuitry disposed on the CGM may control the communication device and be operable to implement the process 400 and communicate with the wearable drug delivery device to cause the wearable drug delivery device to suspend or increase delivery of insulin.

The described system and processes may also be useful in the detection and mitigation of a condition known as diabetic ketoacidosis, which is a condition caused by elevated ketones in the blood of a user. Diabetic ketoacidosis is a condition in which glucose is unavailable to the body for energy and the body begins to burn fat for energy. Ketones are chemicals that the body creates when it breaks down fat for energy. By monitoring trends in a user's ketone values (i.e., ketone value trend) according to the processes described herein, onset of ketoacidosis may be avoided. Another example of an analyte sensor (described with reference to later example) may be a ketone sensor. Note that ketones may also be detected using a breath sensor (which is not shown but may be incorporated in a controller shown in a later example) or urine content sensor; however, a subcutaneous ketone sensor gives more accurate information and is more continuous.

In block 402, the logic circuitry may be operable to implement the process 400 and set the detection rate of the sensor based on a selection from multiple detection rates. The logic circuitry may be operable to execute programming code stored in a memory that includes a lookup table with multiple detection rates and criteria that informs the logic circuitry of which respective detection rate to select from the multiple detection rates.

In block 404, the logic circuitry implementing process 400 obtains one or more analyte measurement values from the sensor and determines a rate of change of analyte measurement values received over a period of time. In an example, the analyte sensor may include a memory coupled to the logic circuitry. In the example, the logic circuitry may be operable to retrieve past analyte measurement values in a memory coupled to the logic circuitry, analyze the analyte measurement value with reference to the past analyte measurement values stored in the memory, determine an updated rate of change of the analyte measurement value and the past analyte measurement values.

In block 406, process 400 determines, based on the determined rate of change, delivery of a liquid drug is to be suspended. In the example of an updated rate of change, the logic circuitry may be further operable to determine whether the updated rate of change exceeds a sample threshold. The sample threshold may be determined according to Equation 3 above or may include multiple analyte measurement values. In response to the updated rate of change exceeding the sample threshold, the logic circuitry may cause the generation of the suspension signal at block 408.

In block 408, the logic circuitry generates a suspension signal. For example, the logic circuitry may generate the suspension signal that may include a duration of the suspension or indicate a treatment protocol for ending the suspension and gradually readministering insulin doses (e.g., end suspension, administer X units or mL of insulin every hour for next two hours, or the like).

In block 410, the logic circuitry may cause the communication circuit to transmit the suspension signal. For example, the logic circuitry may send a control signal to the communication circuit which establishes a communication session with a communication device of the wearable drug delivery device to receive the suspension signal.

In addition, the logic circuitry, when determining the rate of change may be further operable to determine a negative rate of change of the analyte measurement values, and in response to the determined negative rate of change, increase the detection rate of the sensor. The logic circuitry may be further operable to, in response to the increased detection rate of the sensor, cause the communication circuit to output a result of a detection of the analyte measurement value from the blood sample of the user to an external device. The output of the result of the detection may be at a rate matching the increased detection rate of the sensor.

Conversely, the logic circuitry may be further operable to determine a positive rate of change of the analyte measurement values, and in response to the determined negative rate of change, decrease the detection rate of the sensor. The logic circuitry is further operable to, in response to the decreased detection rate of the sensor, cause the communication circuit to output a result of a detection of the analyte measurement value from the blood sample of the user to an external device. The output of the result may be at a rate matching the decreased detection rate of the sensor.

In another example, the logic circuitry may be further operable to provide, based on the set detection rate, the analyte measurement value to the communication circuit for transmission by the communication circuit.

Other technical features may be readily apparent to one skilled in the art from the following figures, descriptions, and claims.

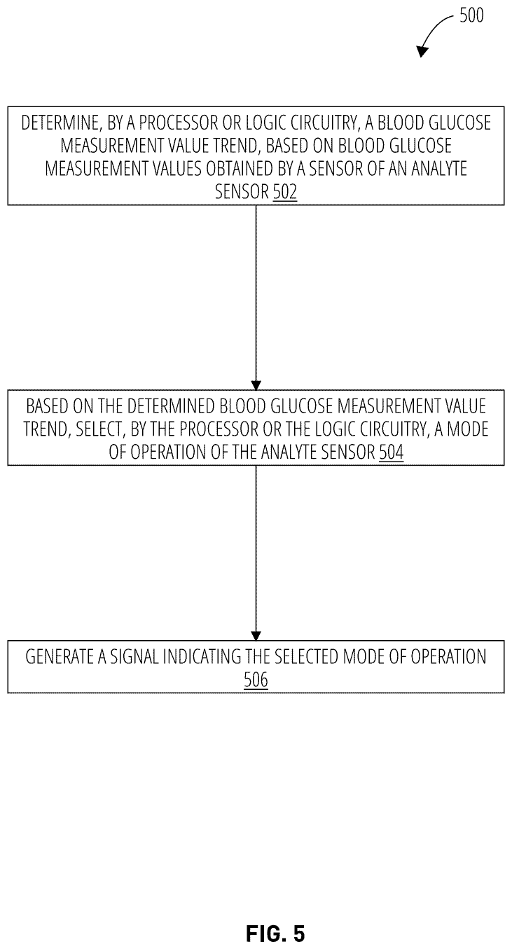

FIG. 5 illustrates an example of a process according to an aspect of the disclosed subject matter. In the process 500, a processor or logic circuitry is operable to respond to events other than or in addition to a change in the rate of change or blood glucose measurement value trend. In the example process 500, the AGC application may be executed by a processor of a wearable drug delivery device or logic circuitry of an analyte sensor. For example, the logic circuitry of the CGM may be operable to respond to rapid blood glucose measurement value changes by communicating alarms and insulin requests to the wearable drug delivery device. It is envisioned that the CGM may have different modes of operation.

In block 502, the processor or the logic circuitry implementing process 500 may be operable to determine that an event affecting a blood glucose measurement value trend of a user has occurred. An event may be ingestion of a meal, participation in exercise, a bolus dosage, sleeping, waking, or the like. The events may be self-reported by a user. For example, the user may request a meal bolus, which is an indication of consumption, or imminent consumption, of a meal. Or, the user may input event information, such as they are about to participate in a 5 kilometer run or other exercise. Alternatively, or in addition, the AGC application may be operable to automatically respond to an event. For example, the AGC application may be operable to implement other features or receive information from other applications via a plug-in or an application programming interface. Examples of the other applications or features may be a fitness application, global positioning applications, meal planning applications, carbohydrate estimating or calorie counting applications, a calendar application, exercise detection or exercise determination applications, and the like. Events indicated by the other applications may be breakfast, lunch or dinner appointments, exercise events (such as a scheduled fitness class and its location), an exercise determination, a location of a restaurant or eatery, or the like. The respective applications, that are operable to provide indications of occurrences of events that may trigger the AGC application to modify a set time interval (or sampling rate) for the CGM to provide readings.

In block 504, the processor or logic circuitry, based on the occurrence of the event (also referred to as “the determined event”), may be operable to select a mode of operation of the analyte sensor. The CGM may have multiple modes of operation that may be set by an external device via a control signal or by logic circuitry operating in the CGM.

For example with reference to FIG. 1A, in a first mode, the CGM may be operable to collect a sample from a user nearly continuously and have sufficient power to output the results of processing the sample, every few seconds or at intervals smaller than the 5 minute intervals shown by times 102-110 of FIG. 1A, to a wearable drug delivery device. In another mode, the CGM may collect samples at times that correspond to a set interval, such as the 5 minute intervals of the times 102-110 of FIG. 1A. The interval may be set at an initial setting (e.g., during a setup procedure), or may be a default setting, and may continue at that setting unchanged until the CGM needs to be replaced. In yet another mode, the CGM may include logic circuitry that is operable to allow the interval to be set at an initial setting, but is also operable to receive a signal from an external device that the logic circuitry interprets as a command signal to alter the interval to, for example, output blood glucose measurement values, such as 112, 114 and 116. Other modes may also be provided, such as a staggered mode of operation in response to negative rate of change or meal bolus (e.g., change to 4 minute reporting, then 2 minute reporting, and then 1 minute reporting) or in response to a positive rate of change or participation in exercise (e.g., change from 1 minute reporting to 3 minute reporting, to 4 minute reporting, then 5 minute reporting). For ease of discussion, all of the other modes are not discussed in detail.

In the case of a recent meal event or a large meal bolus, the AGC application may be operable to request on-demand CGM readings to ensure if more adjustments to basal is needed to compensate for the rising blood glucose. An on-demand CGM reading may be whenever the AGC application requests a reading (i.e., a blood glucose measurement value). For example, when the AGC application determines that the user's blood glucose is within the target range for a predetermined amount of time, such as 20 minutes or the like, the AGC application may request that the CGM provide a reading every 5 minutes. Conversely, if the AGC application determines that a negative rate of change is occurring, the AGC application may request a reading every 2 minutes depending upon the degree of the negative rate of change. For example, a steeper negative rate of change may cause the AGC application to request more frequent reading reports, such as every 1 minute or the like. An event may also be a time of day as it may be beneficial to sample more frequently during particular times of day, such as during mealtimes, and less frequently during sleep times. Such tailored sampling by the processor or the logic circuitry may preserve the power supply (e.g., battery life) and/or sensor life of the CGM.

In block 506, the processor of the wearable drug delivery device or the logic circuitry of the CGM may be operable to generate a signal indicating the selected mode of operation.

In an example of the processor of the wearable drug delivery device executing process 500, the processor may cause a communication device to establish a communication session, if one is not already established, with the CGM and the logic circuitry. The processor may output a control signal that indicates the selected mode of operation based on the determined event. In an example, a communication device of the wearable drug delivery device may transmit the control signal via the established communication session to the logic circuitry of the CGM. The logic circuitry of the CGM may respond to the control signal by changing the mode of operation to the selected mode indicated by the control signal.

Alternatively, the logic circuitry may be executing process 500, the logic circuitry may cause a communication device to establish a communication session, if one is not already established, with the wearable drug delivery device A process of the logic circuitry may output a signal that indicates the selected mode of operation, and another process of the logic circuitry may respond based on the determined event. In an example, a communication device of the wearable drug delivery device may transmit the control signal via the established communication session to the logic circuitry of the CGM. The logic circuitry may respond to the control signal by changing the mode of operation to the selected mode indicated by the control signal.

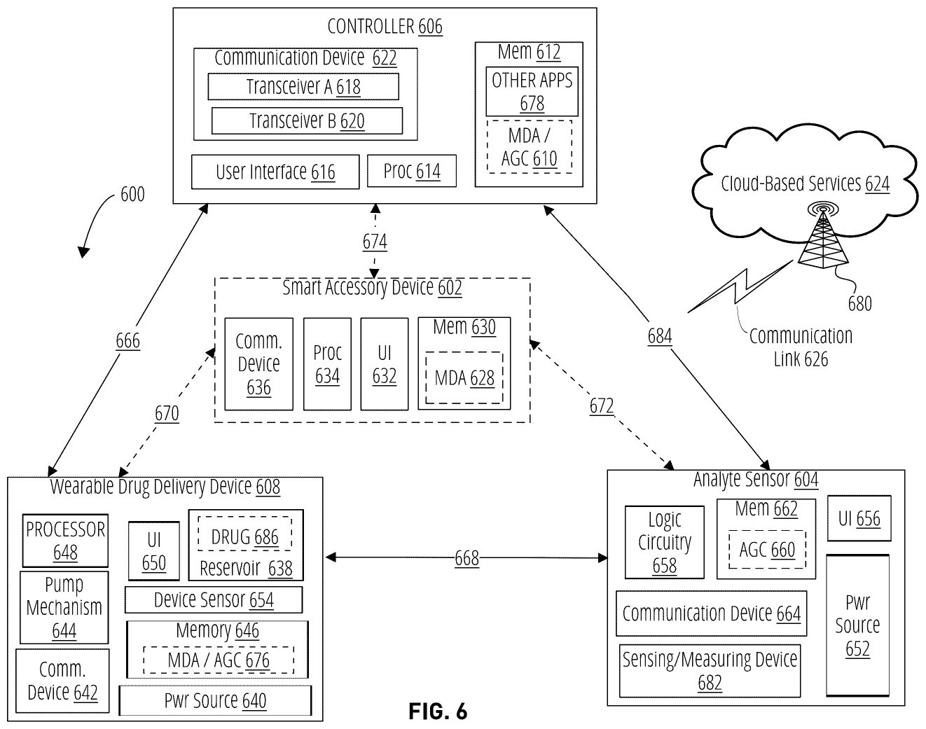

FIG. 6 illustrates a functional block diagram of a system example suitable for implementing the example processes and techniques described herein.

The automatic wearable drug delivery system 600 may implement (and/or provide functionality for) a medication delivery algorithm (MDA), such as an artificial pancreas (AP) application or an automatic glucose control (AGC) application, to govern or control automated delivery of a drug, a therapeutic, or a medication, such as insulin, to a user (e.g., to maintain euglycemia-a normal level of glucose in the blood). The automatic wearable drug delivery system 600 may, for example, include an analyte sensor 604, a controller 606, a wearable drug delivery device 608, and an optional smart accessory device 602.

The controller 606 may be remote from the wearable drug delivery device 608 and may include a user interface 616, a communication device 622, a memory 612, and a processor 614. The user interface 616 is coupled to the processor 614 and operable to receive inputs related to a physiological condition of a user and provide the input to the processor 614. In an example, the input may be a request for a bolus dosage. The controller 606 may include a user interface 616, which may be a keypad, a touchscreen display, levers, light-emitting diodes, buttons on the controller 606, a microphone, a camera, a speaker, a display, or the like, that is configured to allow a user to enter information and allow the controller 606 to output information for presentation to the user (e.g., alarm signals, exercise recommendations (e.g., exercise times and/or exercise intensity, and the like). The user interface 616 may provide inputs, such as a voice input, a gesture (e.g., hand or facial) input to a camera, swipes to a touchscreen, or the like, to processor 614 which the programming code interprets.

The controller 606 may be a computing device such as a smart phone, a tablet, a personal diabetes controller, a dedicated diabetes therapy controller, or the like. In an example, the controller 606 may include a processor 614, a controller memory 612, a user interface 616, and a communication device 622. The controller 606 may contain analog and/or digital circuitry that may be implemented as a processor 614 for executing processes based on programming code stored in the controller memory 612, such as the medication delivery algorithm or application or an automatic glucose control application (MDA/AGC) 610 and related programming code as well as sampling threshold values. The processor 648 may be used to program, adjust settings, and/or control operation of the wearable drug delivery device 608 and/or the analyte sensor 604 as well as the optional smart accessory device 602.

The one or more transceivers, transceiver A 618 and transceiver B 620 may operate according to one or more radio-frequency protocols. In the example, the transceivers 618 and 620 may be a cellular transceiver and a Bluetooth® transceiver, respectively. For example, the transceiver A 618 or transceiver B 620 may be configured to receive and transmit signals containing information usable by the MDA/AGC 610.

The wearable drug delivery device 608 may include processor 648, a reservoir 638, a communication device 642, a power source 640, a memory 646, device sensor 654, user interface (UI) 650, and a pump mechanism 644. The processor 648 may be operable to control the drug delivery device. The reservoir 638 may be configured to contain a liquid drug. The communication device 642 may be coupled to the processor 648. The pump mechanism 644 may be responsive to the processor 648 and fluidically coupled to the reservoir 638.

The memory 646 may store programming code executable by the processor 648. The programming code, for example, may enable the processor 614 to control expelling insulin from the reservoir 638 in response to control signals from the controller 606 and MDA/AGC 610 or based on signals from the optional MDA/AGC 676.

In the example, the communication device 642, which may be a receiver, a transmitter, or a transceiver or other circuitry that operates according to one or more radio-frequency protocols, such as Bluetooth, Wi-Fi, a near-field communication standard, a cellular standard, or the like. The communication device 642 may enable the processor 648 to communicate with the controller 606 and the analyte sensor 604.

The wearable drug delivery device 608 may be attached to the body of a user, such as a patient or diabetic, at an attachment location and may deliver any therapeutic substance to a user at or around the attachment location. For example, a bottom surface of the wearable drug delivery device 608 may include an adhesive to facilitate attachment to the skin of a user as described in earlier examples.

The reservoir 638 may store liquid drugs, medications or therapeutic agents suitable for automated delivery, such as diabetes treatment drugs (e.g., insulin, glucagon, glucagon-like peptides), pain relief drugs (e.g., morphine), hormones, blood pressure medicines, chemotherapy drugs, or the like, such 686. The wearable drug delivery device 608 may include a needle or cannula (not shown) coupled to the reservoir 638 and extending into the body of the user for delivering a liquid drug into the user's body of the user (which may be done subcutaneously, intraperitoneally, or intravenously), and a pump mechanism 644 under control of the processor 648 that is operable to transfer the liquid drug from the reservoir 638 through the needle or cannula and into the user.

The power source 640, such as a battery, a piezoelectric device, other forms of energy harvesting devices, or the like, for supplying electrical power to the pump mechanism 644 and/or other components (such as the processor 648, memory 646, and the communication device 642) of the wearable drug delivery device 608.

In some examples, the wearable drug delivery device 608 may include a user interface 650, which may be a keypad, a touchscreen display, levers, light-emitting diodes, buttons on a top portion or side portion of the drug delivery device 608, a microphone, a camera, a speaker, a display, or the like, that is configured to allow a user to enter information and allow the drug delivery device 608 to output information for presentation to the user (e.g., alarm signals or the like). The user interface 650 may provide inputs, such as a voice input, a gesture (e.g., hand or facial) input to an optical sensor, swipes to a touchscreen, or the like, to processor 648 which the programming code interprets.

The wearable drug delivery device 608 may also include a device sensor 654 that may include an accelerometer, a gyroscope, a skin conductance measuring device (e.g., to measure perspiration due to exercise), or the like. The device sensor 654 may be coupled to and provide inputs to the processor 648.

The smart accessory device 602 may be a smart watch, another wearable smart device, including eyeglasses, provided by other manufacturers, a global positioning system-enabled wearable device, a wearable fitness device, smart clothing, or the like, and may be operable to communicate with the other components of system 600 via wireless communication links 670, 672, or 674.

For example, the smart accessory device 602 may include a communication device 636, a processor 634, a user interface 632 and a memory 630. The user interface 632 may be a graphical user interface presented on a touchscreen display of the smart accessory device 602. The memory 630 may store programming code to operate different functions of the smart accessory device 602 as well as an instance of the MDA 628. The processor 634 that may execute programming code, such as MDA 628 for controlling the wearable drug delivery device 608 to implement the processes and techniques of FIGS. 1-4 described herein.

The analyte sensor 604 may include logic circuitry 658, a memory 662, a sensing/measuring device 682, a user interface 656, a power source 652, and a communication device 664. The analyte sensor 604 may be configured to detect multiple different analytes, such as lactate, ketones, uric acid, sodium, potassium, alcohol levels, blood glucose, proteins, hormones, or the like, and output results of the detections, such as measurement values or the like, for receipt by one or more of 602, 606 or 608. The analyte sensor 604 may be operable to receive information from a breath sensor or a urine sensor.

The logic circuitry 658 of the analyte sensor 604 may be operable to perform many functions. For example, the programming code stored in the memory 662 may enable the logic circuitry 658 to manage the collection and analysis of data detected by the sensing and measuring device 682, such as blood glucose measurement values, providing trend information and the like. The memory 630 may be configured to store information and programming code, such as an instance of the AGC 660. The logic circuitry 658 may include discrete, specialized logic and/or components, an application-specific integrated circuit, a microcontroller or processor that executes software instructions, firmware, programming instructions, such as the AGC 660, stored in the memory 662), or any combination thereof. The logic circuitry 658 may be operable to implement and provide the functions described with reference to the examples of FIGS. 2, 4 and 5.

In an example, the analyte sensor 604 may be a blood glucose monitor removably attachable via adhesive, for example, to a body of the user. In such an example, the analyte sensor 604 is operable to measure a blood glucose measurement value of the user (not shown) and communicate with the controller 606 and the drug delivery device 608 via the communication device 664 under the control of the logic circuitry 658. The logic circuitry 658 may be operable to execute the AGC 660 that enables implementation of the processes 200, 400 and 500 described above.

The analyte sensor 604 may, in an example, be operable provide blood glucose measurement values at selected set time intervals, such as 5 minutes, 4 minutes, 3 minutes, 2 minutes, 1.5 minutes, 1 minute, 30 seconds or near continuously, depending upon the selected setting. For example, at an initial setting of the AGC 660, the logic circuitry 658 of analyte sensor 604 may be operable to sample a user's blood glucose at a predetermined time interval, such as every 5 minutes, or the like, and output a blood glucose measurement value. The initial setting of the set time interval for the sensing/measuring device 682 to take samples and make measurements may be made via the user interface 656 or in response to control signals from the controller 606 or the wearable drug delivery device 608. In an example, a graphical user interface may be presented that enables selection of set time interval (e.g., 1 minute) from a number of set time intervals, such as those listed above.

The logic circuitry 658 upon execution of the AGC 660 to provide the functions describe with reference to FIGS. 1-5 above may be operable to for a period of time, receive a blood glucose measurement value from the analyte sensor 604 via communication link 668 at a set time interval within the period of time. The period of time may be, for example, 30 minutes, 90 minutes, 120 minutes, 24 hours, 36 hours or 96 hours. The logic circuitry 658 may be further operable to determine a rate of change of the blood glucose measurement values received from the analyte sensor over the period of time. Based on the determined rate of change, the logic circuitry 658 may be operable to select a different set time interval stored in memory 662. For example, a look up table containing a list of different set time intervals may be maintained in memory 662. The processor 648 of the wearable drug delivery device 608 may also perform the described functions with the memory 646 also maintaining a look up table containing a list of different set time intervals.

The communication device 664 of analyte sensor 604 may have circuitry that operates as a transceiver for communicating the blood glucose measurement values to the controller 606 over the wireless link 684 or with the wearable drug delivery device 608 over the wireless communication link 668.

Services provided by cloud-based services 624 may include data storage that stores anonymized data, such as blood glucose measurement values, data related to set time intervals for analyte sensors produced by different manufacturers, and other forms of data. The cloud-based services 624 may be accessed via data network device 680, which may be a Wi-Fi device, a cellular communication tower, a local area network, a campus wide network or the like.

The wireless communication links 666, 668, 670, 672 and 684 may be any type of wireless link operating using known wireless communication standards or proprietary standards. As an example, the wireless communication links 666, 668, 670, 672 and 684 may provide communication links based on Bluetooth®, Zigbee®, Wi-Fi, a near-field communication standard, a cellular standard, or any other wireless protocol.

Software related implementations of the techniques described herein, such as the processes examples described with reference to FIG. 2, FIG. 4 and FIG. 5 may include, but are not limited to, firmware, application specific software, or any other type of computer readable instructions that may be executed by one or more processors or logic circuitry. While the processes 200, 400 and 500 were primarily discussed as being implemented on a wearable drug delivery device 608 or an analyte sensor 604, a processor of controller 606 may also be operable to provide the above functions and take the described actions. The computer readable instructions may be provided via non-transitory computer-readable media. Hardware related implementations of the techniques described herein may include, but are not limited to, integrated circuits (Ics), application specific Ics (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed MDA/AGC application and algorithms may be operable to perform various functions related to open loop operations, such as the generation of prompts requesting the input of information such as weight or age. Similarly, a dosage amount of insulin may be received by the MDA/AGC application or algorithm from a user via a user interface. Other open-loop actions may also be implemented by adjusting user settings or the like in an MDA/AGC application or algorithm.

Some examples of the disclosed device or processes may be implemented, for example, using a storage medium, a computer-readable medium, or an article of manufacture which may store an instruction or a set of instructions that, if executed by a machine (i.e., processor, logic circuitry, or controller), may cause the machine to perform a method and/or operation in accordance with examples of the disclosure. Such a machine may include, for example, any suitable processing platform, computing platform, computing device, processing device, computing system, processing system, computer, processor, logic circuitry, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor, or logic circuitry, when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, logic circuitry, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A wearable drug delivery device, comprising:
a processor;
a memory storing an automatic glucose control application, programming code, and data related to the automatic glucose control application;
a reservoir shaped to contain a liquid drug;
a pump mechanism controlled by the processor and operable to deliver the liquid drug; and
a communication circuit controlled by the processor and operable to communicate with an external device, wherein:
the processor when executing the automatic glucose control application is operable to:
for a period of time, receive blood glucose measurement values at a first set time interval within the period of time;
determine a missed blood glucose measurement value; and
based on the determined missed blood glucose measurement value, select a second set time interval different than the first set time interval.

2. The wearable drug delivery device of claim 1, wherein the processor is configured to:
estimate an expected blood glucose measurement value based on the received blood glucose measurement values; and
determine the missed blood glucose measurement value based on the expected blood glucose measurement value.

3. The wearable drug delivery device of claim 1, wherein the second set time interval is about one minute to about five minutes.

4. The wearable drug delivery device of claim 1, wherein the processor is further configured to determine a rate of change of the blood glucose measurement values.

5. The wearable drug delivery device of claim 4, wherein the processor is configured to select the second set time interval based on the determined rate of change.

6. The wearable drug delivery device of claim 5, wherein the processor is configured to select the second set time interval from a table, wherein the table is based on a determined rate of change, wherein the second set time interval is a set time value that is half, one third, one quarter or one fifth of the first set time interval.

7. The wearable drug delivery device of claim 1, wherein the processor is configured to:
compare at least one of the received blood glucose measurement values to a threshold; and
select the second set time interval based on the comparison.

8. The wearable drug delivery device of claim 1, wherein the period of time spans multiple set time intervals.

9. The wearable drug delivery device of claim 1, wherein the processor when executing the automatic glucose control application is further operable to:
determine that delivery of the liquid drug is to be suspended based on the determined missed blood glucose measurement value; and
generate a suspension signal to cause suspension of delivery of the liquid drug.

10. The wearable drug delivery device of claim 1, wherein the communication circuit is further operable to:
establish, in response to a control signal from the processor, a communication session with an analyte sensor remote from the wearable drug delivery device; and
receive the blood glucose measurement value during the communication session, wherein the communication session is established to enable receipt of the blood glucose measurement value at the set time interval.

11. A computer-implemented method of automatic glucose control, comprising:
for a period of time, receiving blood glucose measurement values at a first set time interval within the period of time;
determining a missed blood glucose measurement value; and
based on the determined missed blood glucose measurement value, selecting a second set time interval different than the first set time interval.

12. The computer-implemented method of claim 11, further comprising:
estimating an expected blood glucose measurement value based on the received blood glucose measurement values; and
determining the missed blood glucose measurement value based on the expected blood glucose measurement value.

13. The computer-implemented method of claim 11, wherein the second set time interval is about one minute to about five minutes.

14. The computer-implemented method of claim 11, further comprising determining a rate of change of the blood glucose measurement values.

15. The computer-implemented method of claim 14, wherein selecting the different set time interval comprises selecting the second set time interval based on the determined rate of change.

16. The computer-implemented method of claim 15, wherein selecting the second set time interval comprises:
choosing the second set time interval from a table, wherein the table is based on the determined rate of change, wherein the second set time interval is a set time value that is half, one third, one quarter or one fifth of the first set time interval.

17. The computer-implemented method of claim 11, further comprising:
comparing at least one of the received blood glucose measurement values to a threshold; and selecting the second set time interval based on the comparison.

18. The computer-implemented method of claim 11, further comprising determining that delivery of the liquid drug is to be suspended based on the determined missed blood glucose measurement value; and
generating a suspension signal to cause suspension of delivery of the liquid drug.

19. The computer-implemented method of claim 11, further comprising:
establishing a communication session with an analyte sensor; and
receiving the blood glucose measurement values during the communication session, wherein the communication session is established to enable receipt of the blood glucose measurement values at the first set time interval.

20. The computer-implemented method of claim 11, wherein selecting the second set time interval comprises calculating the second set time interval based on a determined rate of change and the first set time interval by dividing the first set time interval by a constant.
